(19) United States
(12) Patent Application Publication
(10) Pub. No.: US 2004/0249255 A1
(43) Pub. Date: Dec. 9, 2004

(54) Hand held tonometer including optical procimity indicator

(76) Inventors: James Robert Arnold Matthews, Berkshire (GB); John Horace Fisher, Hertfordshire (GB); Paul Antony Merritt, Hertfordshire (GB); Neil Anthony Atkins, Berkshire (GB); David Philip Graham, Hampshire (GB)

Correspondence Address: BARNES & THORNBURG, P.O. BOX 2786, CHICAGO, IL, 60690-2786, US

(21) Appl. No.: 10/486,417

(22) PCT Filed: Aug. 13, 2002
(86) PCT No.: PCT/GB02/03729

(30) Foreign Application Priority Data
Aug. 14, 2001 (GB) 0119741.7
Aug. 14, 2001 (GB) 0119743.3
Aug. 14, 2001 (GB) 0119744.1
Aug. 14, 2001 (GB) 0119746.6

(51) Int. Cl. A61B 3/16

(52) U.S. Cl. 600/401

(57) ABSTRACT

A puff tonometer includes two light sources located at diametrically opposite points, preferably equidistant from the optical axis of the objective lens assembly of the tonometer. The sources are arranged to direct light forwardly of the tonometer such that, in use, and when positioned close to a patient's eye under test, light from the two sources, after reflection by the anterior corneal surface of the eye under test, will be imaged by the objective lens assembly of the tonometer, to appear as two small areas of light in the field of view. The spacing and position of the two supplementary light sources relative to the objective lens assembly are selected so that as the tonometer is moved towards an eye under test and begins to approach the distance from the eye at which the tonometer will automatically discharge a puff of air towards the eye, the light reflected by the corneal surface will appear as two closely spaced spots of light which, with continued movement of the unit towards the eye, will begin to move away from each other, and will be replaced by two segments of light reflected from the corneal surface of the eye, which reflected light also becomes focused onto an array of photoelectric detectors as the unit approaches the critical firing distance from the eye. The light from the two supplementary light sources is coloured and is distinct from the light used to form the two segments and when in focus trigger the air discharge. If the latter light is red, the light from each of the two supplementary light sources may be green or blue for example. When equidistant the position of the two spots of light relative to the centre of the field of view readily indicates to the user whether the unit is centered on the eye. A resilently deformable extension may be fitted to the tonometer nozzle whose natural length is greater than the critical distance of the front of the nozzle from the eye at which an air pulse is released, and which can be pushed against the patient's face and thereby reduced in length until the critical distance is reached.

### FIELD OF INVENTION

This invention relates to a non-contact air impulse tonometer of the type in which a controlled pulse of air is directed towards the cornea of an eye under test and the resulting momentary deformation of the cornea monitored, to determine the internal pressure of the eye relative to the ambient, and indicate the monitored pressure to the user.

### BACKGROUND TO THE INVENTION

An air impulse tonometer which can be held in the hand in use, is described in UK 2175412 and EP0289545. Such a tonometer will be referred to as a tonometer of the type described.

Initial alignment of such a tonometer with an eye under test, can be difficult since the optical system developed for that tonometer includes an eyepiece which does not allow an image of the eye under test to be seen by the user when looking through the eyepiece. Instead a source of illumination typically comprising a filament lamp and red filter, is followed by a condenser lens and objective lens assembly, to project light from a source, (typically red light) through a mask (containing two windows but otherwise obscured) towards the eye. At one particular distance between the eye under test and the objective lens assembly, the convex anterior surface of the cornea of the eye and the objective lens form an in-focus image of the two windows which can be seen by a user looking through the eyepiece, the windows appearing as two separate segments (red if red light is employed). Since the focus of the light from the source (e.g. red light) is determined by the distance between the optical system in the hand held unit and the anterior corneal surface of the eye under test (from which it is reflected), movement of the unit towards and away from the eye will alter the focus of the two illuminated segments (typically of red light) as seen by the user, thereby assisting the user in positioning the unit relative to the eye.

The sensing mechanism is set up to instigate an air pulse when the reflected light is centred on the optical axis and an image of the mask is in focus on a plurality of photoelectric sensors and each receive preselected amounts of reflected light. This also corresponds to the position of the unit relative to the eye at which the two illuminated segments are in focus in the field of view.

In practice the user will tend to look along the side of the unit as he/she moves the unit into position until he/she is satisfied that, from experience, the unit is nearly close enough to the eye to allow the measurement to be taken. At this point the user can now look through the eyepiece of the unit to view the image in the field of view, as described above, to position the unit into the firing position.

If the user moves the unit closer to the eye before the air pulse is initiated, the two illuminated segments (typically of red light) will begin to go out of focus again (having previously become in-focus at the correct distance), and further movement of the unit towards the eye will result in the filament of the lamp coming into focus in the field of view. Should this happen the user knows to move the unit backwards until the correct point of focus is achieved once again, whereupon it may be necessary to move the unit from side to side or up and down to centre it on the eye, before the unit will fire.

### OBJECT OF THE INVENTION

It is an object of the present invention to provide a mechanism by which the user is further assisted in positioning the unit relative to the eye of a patient, so as to cause the unit to trigger and fire a puff of air towards the eye.

### SUMMARY OF THE INVENTION

According to one aspect of the present invention two small light sources are located at diametrically opposite points, typically equidistant, from the optical axis of the objective lens assembly of a tonometer of the type described, such that in use and positioned close to a patient's eye under test, light from the two sources, after reflection by the anterior corneal surface of the eye under test, will be collected by the objective lens assembly of the tonometer, to appear as two areas of light in the field of view.

The spacing and position of the two light sources relative to the objective lens assembly are selected so that as the unit is moved towards an eye under test and begins to approach the critical distance from the eye at which firing is to be triggered, the light reflected by the corneal surface will appear as two closely spaced spots of light which, with continued movement of the unit towards the eye, will begin to move away from each other, and in the case of a tonometer of the type described, will be replaced by two areas of light corresponding to the two mask windows as the unit approaches the critical firing distance from the eye.

Seeing the two spots of light in the field of view, ahead of the two areas of light from the main source of illumination, assists the user in knowing that the unit under his/her control is approaching the eye under test but still needs to be moved towards the patient.

Preferably the light from the two supplementary sources is coloured and is distinct from that from the main source, and where the source of illumination is red, light from each of the two small supplementary sources may be green. However it need not be the same and one may be green and the other blue or yellow for example.

The position of the two spots of light relative to the field of view will also tell the user whether the unit is centred on the eye. Thus for example if the sources are equidistant and the spots are not symmetrically located in the field of view, and do not lie on a straight line passing through that central region of the field of view, the optical axis of the unit is probably not centred on the eye. Movement of the unit to the left or the right (and/or up or down if the spots are too low or too high) will attain the desired adjustment, enabling the user to then move the unit in a forward direction in the knowledge that it is correctly centred on the eye under test.

Preferably the two small light sources are positioned so that light therefrom is directed towards the anterior corneal surface of the eye, such that when the latter is at a distance from the tonometer which is just greater than the critical distance at which firing will occur, two distinct spots of light will be visible in the field of view and will move apart and disappear and be replaced by the light from the source of illumination which illuminates the two mask windows as the unit is moved closer to the eye.

Preferably the light from these two light sources is of a different colour from the other light images which appear in the field of view during use.

In particular it is very desirable that the wavelength of the light from the two small light sources is significantly different from that of the main source of illumination, and the photo-sensors are selected so as to have a peak response to the wavelength of the light from the main source and a minimal or zero response at the wavelength of the light from the two small supplementary light sources, so that light from the latter which may reach the photoelectric sensors does not significantly affect the output of the sensors.

Typically the two small sources comprise two LED's.

Preferably lens-capped LED's are used the focusing effect of the integral lenses serving to concentrate the light therefrom towards the eye under test. If the LED's do not include integral lens caps, separate miniature lenses may be provided to focus the emitted light as required.

Power for the LED's may be obtained from a power supply associated with the tonometer unit.

An ON/OFF switch may be provided to power the LED's only when required.

Such a switch may be operated by a push button on the unit, located so as to be capable of being pressed by the thumb or a finger of the hand used by the user to hold the tonometer.

Preferably power to the LED's is removed upon the firing of the unit, and the ON/OFF switch may be associated with or be integrated into the RESET switch associated with the unit, which has to be pressed to arm the unit ready to detect an eye and fire an air pulse towards it.

Alternatively the two light sources may comprise two optical fibres leading away from a lamp in the tonometer.

If coloured light is required the optical fibres may be formed from coloured glass or the light path may include a coloured filter.

Preferably the lamp is the filament lamp used to illuminate the mask in the objective lens assembly, with the light for the fibres being obtained from upstream of the red filter.

If angled semi-reflecting surfaces are employed in the tonometer optics, then the two windows of the mask need to be oriented so that the optical path to the photodetectors is the same for each window so that both will be imaged in the same way at the same time.

In order for the light from the supplementary sources to shine through the two windows, following reflection from the patient's cornea, and be seen by the user of the tonometer it is preferable for the supplementary sources to be oriented in a plane going through the centre of the two windows.

Preferably therefore, where the plane semi-reflecting mirrors are angled about a horizontal axis (i.e the axis will be horizontal when the tonometer is held upright), the two points are to the left and right of the objective lens assembly. The LED's or fibre optic ends may be incorporated into the tonometer housing or in lateral enlargements on either side of the tonometer housing.

In order to further assist the user to determine the position needed to achieve firing, an object may be placed in the optical path of the light from the source of illumination in the tonometer such that an in-focus image of this object will be formed in the user's field of view when the unit is at the critical distance from the eye under test, at which firing will occur.

Typically the object is an opaque “hairline” pattern in a transparent support.

The pattern may be formed from a photographic image on a sheet of clear glass or clear plastics material or from an etched metal film on a sheet of glass or plastics. Alternatively it may be formed by etching a metal foil or from wire(s).

Typically the pattern comprises at least one line which extends in a plane generally perpendicular to the axis along which light is projected from the lamp.

The pattern may for example comprise a planar array such as a single line, two lines which cross at an angle, a circular outline with two or more radial lines, or a spiral.

A second object which may be any of the above may be located in the same region of the tonometer as the first object, albeit in a plane which is spaced from the plane containing the first object, on that side thereof which will come into focus in the field of view just before the first object comes into focus, as the unit is moved slowly towards the patient's eye.

Preferably the second object comprises a pattern which is visually distinguishable (as by orientation or content) from the first.

Thus if the objects are single lines and the line which comes into focus at the firing position appears vertical, the wire which is to come into focus earlier is preferably arranged so that it will appear horizontal, or vice versa.

Alternatively if the first object comprises a pair of lines which cross at an angle (say 45° to define a letter X) the second object may comprise a pair of lines which cross at right angles and define a cross.

A third object may also be provided, again preferably distinguishable from both the first and the second objects, at a position relative to the main illumination source such that its image will come into focus if the unit is moved closer to the eye than the critical firing position.

In a tonometer of the type described embodying two additional light sources in accordance with the invention and which includes one or more objects as aforesaid, the eyepiece can be re-designed to give a lower magnification which will give an in-focus image of the patient's eye at a distance via the objective lens.

A tonometer incorporating the re-designed lower magnification eyepiece and the two supplementary light sources and one or more objects as aforesaid allows the user to look through the eyepiece and identify the patient's eye to be tested, whilst at some distance from the patient's face. Thereafter the user can move the unit towards the eye, keeping the image of the eye in the centre of the field of view. As the distance between the unit and the patient's eye decreases, the light from the two sources appears, followed by red light, reflected from the anterior surface of the cornea, which will increase to fill the windows of the mask just prior to the firing position.

As the tonometer is moved further towards the firing position the image of the, or each object, as aforesaid, will be seen, and these can be aligned and focused by appropriate movement of the unit, so that it is finally in the correct alignment position to fire.

In a unit incorporating a lower magnification eyepiece, the latter and objective lens form a simple telescope with an inverted image. A Pechan-Schmidt prism may therefore be located between the eyepiece lens and a window through which the user looks, to invert the image and present to the user an image of the patient's eye which is correctly oriented and handed in a vertical and horizontal sense.

The focal length of the eyepiece may be in the range 62-100 mm, typically 80 mm.

In a tonometer incorporating a modified eyepiece to enable the user to view the patient's eye as aforesaid, the image of the patient's pupil will disappear as the unit is moved closer to the patient and shortly before the unit is close enough for reflected red light from the anterior corneal surface to illuminate the mask. It is in this region that the light from the two diametrically opposed small light sources provided by the present invention is first seen in the field of view to act as a guide, up to when the red light appears.

Where the eyepiece has not been so modified, the user may have to view the patient's face and eye which is to be tested, by looking along the side of the unit prior to adjusting their viewing position to look through the eyepiece once the tonometer is close to the eye, and begin to look for the green light in the field of view, and as a further aid to positioning such a tonometer relative to an eye, a resiliently deformable extension to the nozzle of the tonometer may be provided, the natural length of the extension being greater than the critical distance between the nozzle and the eye under test at which firing will occur, and which can be compressed with minimal force to a length equal to and less than the critical distance, by pushing against the patient's face. If the minimum compressed length is greater than the protrusion of the nozzle beyond the front of the tonometer, the former can never contact the eye.

By positioning the tonometer with the end of the protruding extension around the eye to be checked, and in contact with the patient's face, the user can then look through the eyepiece and gently push the tonometer towards the patient's face so as to compress the extension until the light image(s) appear (if they are not already visible in the field of view), and thereafter adjust the tonometer to centre the light image(s) and achieve the firing position, at which point the pulse of air is released towards the eye and the response is monitored by the unit.

By making the extension of transparent material or with sides cut away to provide viewing slots or as a helix of wire, the user can still see the eye whilst looking along the side of the tonometer to assist in initial alignment of the tonometer, and the light from the two supplementary light sources can also reach the eye.

Alternatively the end of the extension which fits to the nozzle may be enlarged or shaped so as to encompass the two light sources or housing enlargements containing them, whether or not viewing slots are provided or the extension is of transparent material or a wire helix.

Preferably the end which is to engage the patient's face is covered with soft crushable material.

The extension may be removable from the nozzle for replacement or cleaning or sterilising. A covering for the end which is to come into contact with the patient, may be a disposable item, or may be removable for cleaning or sterilising.

The invention will now be described by way of example with reference to the accompanying drawings, in which:

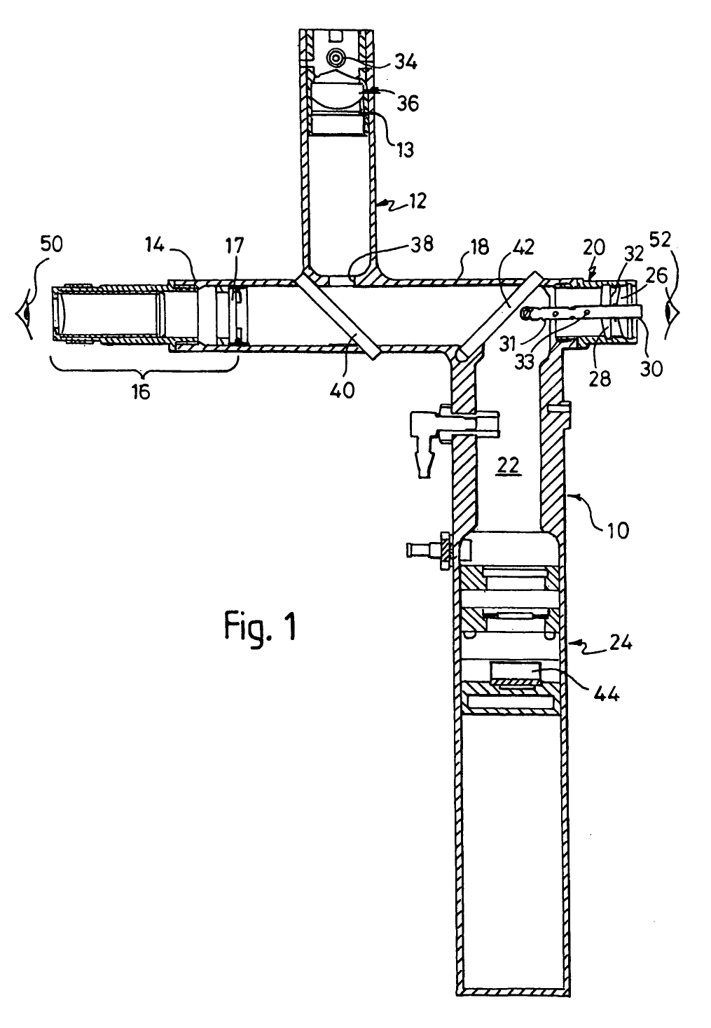

FIG. 1 is a cross-section through the optics and pneumatic chamber of an air impulse tonometer of the type described and can be compared with the drawings in UK 2175412 and EP 0289545,

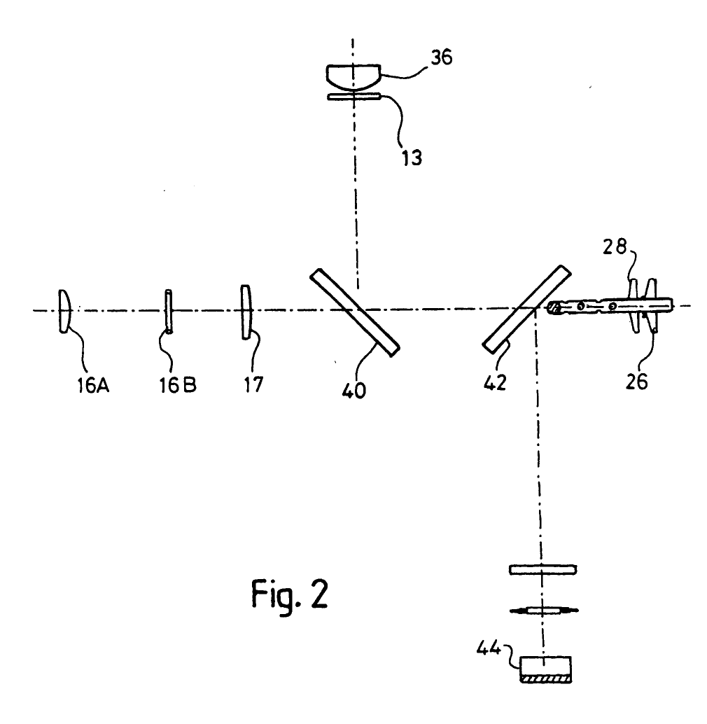

FIG. 2 is a schematic of the optical paths of the device shown in FIG. 1,

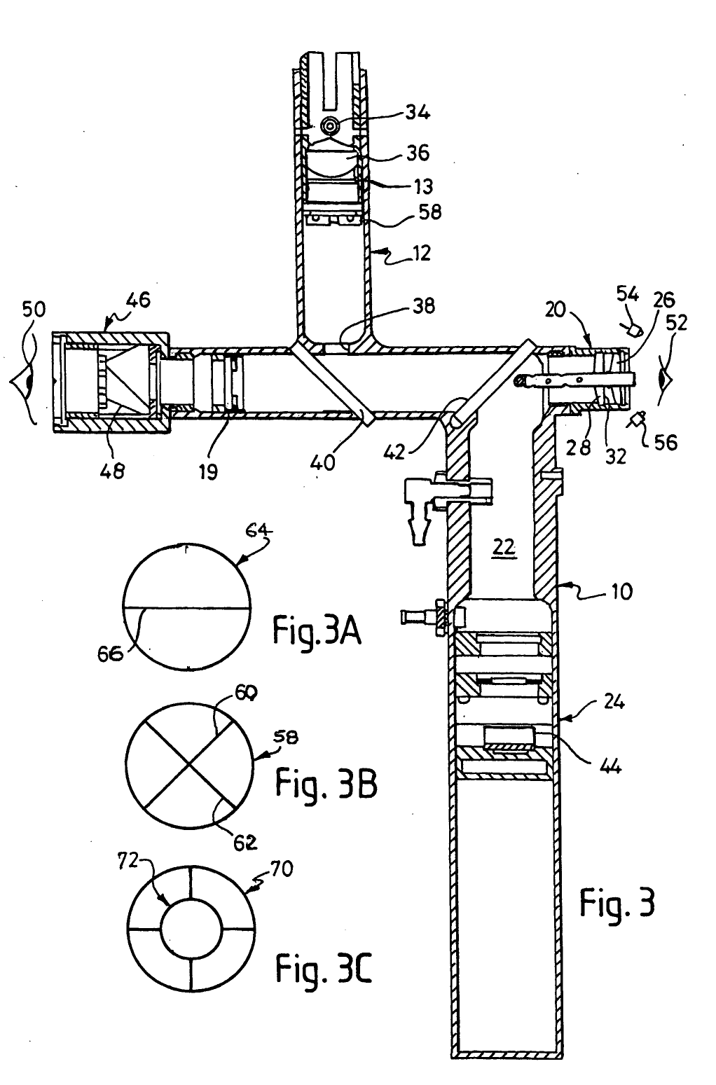

FIG. 3 is a cross-section through an air impulse tonometer similar to that of FIG. 1, but modified to incorporate the present invention and incorporating a modified eyepiece which includes a roof-prism to invert the image,

FIGS. 3A-3C show different hair-line objects for inclusion in the lamp housing,

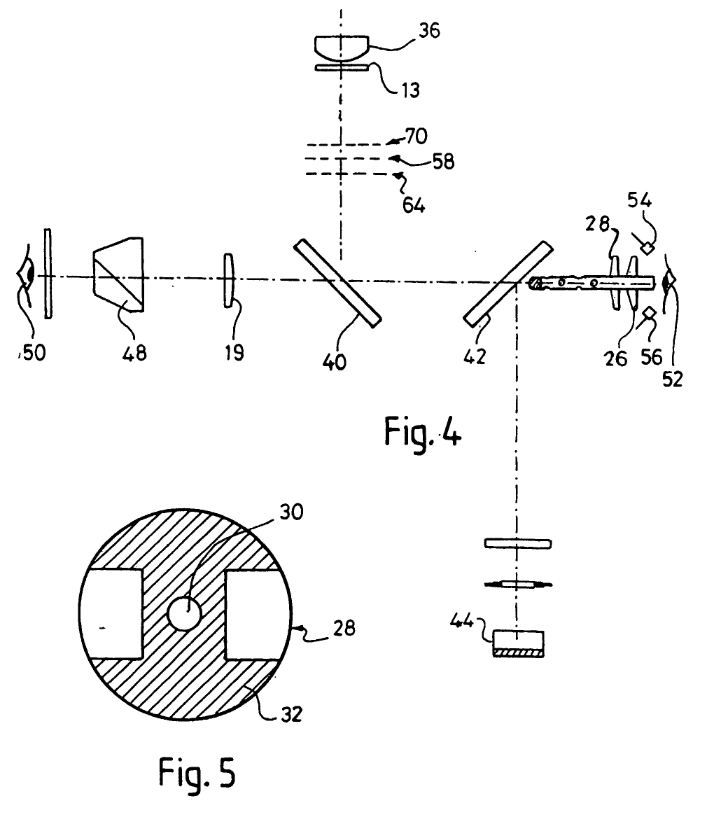

FIG. 4 is a schematic of the optical paths of the device shown in FIG. 3,

FIG. 5 shows the form of the mask on one of the lenses in the final lens assembly,

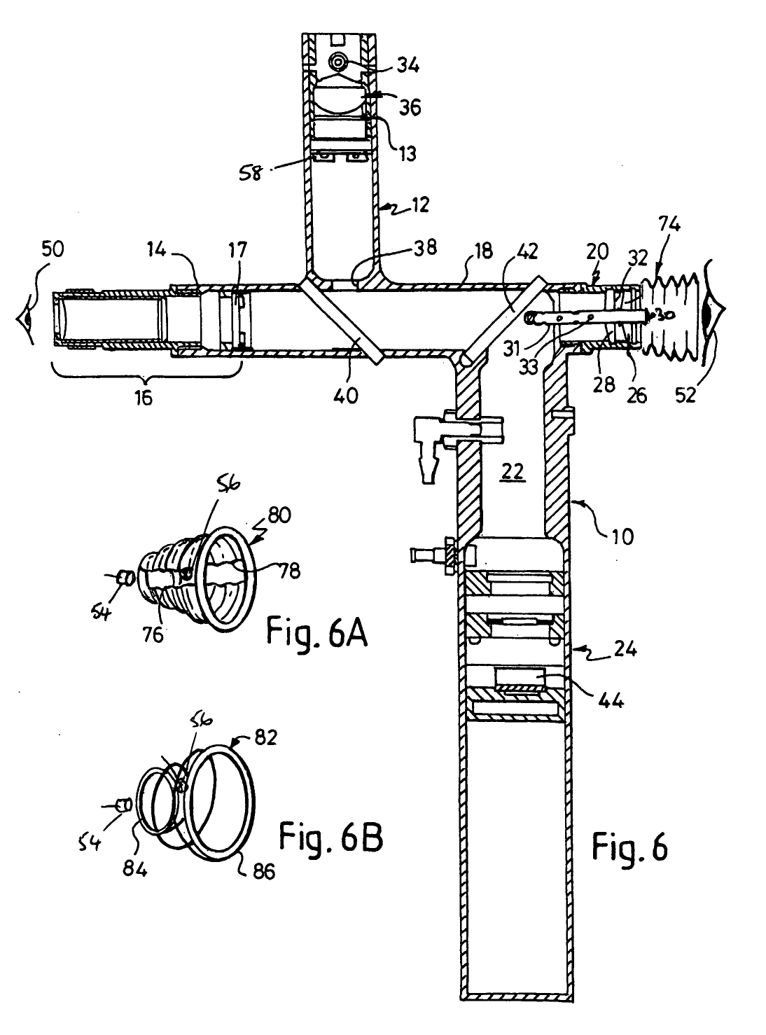

FIG. 6 is a cross-section through a tonometer with a further positioning aid shown on the nozzle, and

FIGS. 6A and 6B show different and preferred forms of construction of the positioning aid.

As shown in FIGS. 1 and 2 a machined chassis 10 comprises a lamp housing 12, a viewing end 14 containing an eyepiece 16 containing a lens 16A, and field stop 16B and field lens 17 (see FIG. 2), a beam splitting section 18, nozzle 20, a plenum chamber 22 and a sensor chamber 24. The tube 20 contains an objective lens assembly 26, 28 and central puff tube 30 supported by the lenses 26, 28 through which it extends. A filter 13 restricts the light transmitted downstream therefrom to wavelengths in the red/infra-red range of the spectrum.

A mask 32 is screen printed onto the face of lens 28, the form of the mask being shown in FIG. 5, as it will appear if viewed axially of the puff tube. The mask includes two windows as shown but is otherwise opaque.

The lamp housing 12 includes a filament bulb 34 from which light is projected as parallel light by a condensing lens assembly 36 to illuminate an aperture 38 at the junction of the housing 12 and the beam splitting section 18. Light passing through 38 is reflected by semi-reflecting mirror 40 towards another semi-reflecting mirror 42 through which it can pass and be focused by the objective lenses 26, 28 onto an eye under test 52. A fraction of the light reflected by the eye and collected by the objective lenses 26, 28 will be reflected by mirror 42 into and through the plenum chamber 22 towards a photoelectric detector assembly 44 in the sensor chamber 24. The remainder will travel through the semi-reflecting mirror 42 and on through the semi-reflecting mirror 40, to the eyepiece 16.

The field lens 17, typically having a focal length of the order of 62 mm, co-operates with the lens 16A in the eyepiece to form an in-focus view of the image of the mask 32 which is formed from the convex curvature of the patient's cornea and the objective lenses 26, 28 to an observer viewing through the eyepiece 16. The lens 16A typically has a focal length of 25 mm. The presence of the mask and puff tube means that the image of the mask, reflected by the patient's eye 52 will, when correctly focused appear as two segments, each similar to a capital letter D, one being a mirror image of the other. The in-focus condition will only occur when the eye is at a particular distance from the end of the puff-tube 30 determined by the focal length of the objective lens assembly 26, 28, and the radius of curvature of the patient's cornea. Typically each of the lenses 26 and 28 is a plano-convex lens having a focal length of the order of 40 mm.

The plenum chamber 22 is pressurised with air when a pulse of air is required. Ignoring the passage leading to the pressure transducer (not shown) the chamber 22 is closed, and air can only escape via the tube 30. The air escapes as a single pulse, the leading edge shape and duration of which is dictated by the geometry of the tube 30 and openings 31, 33, the volume of the plenum chamber 22, the shape and volume of the passage leading to the pressure transducer (not shown), and the volume of the pulse of air introduced into the plenum chamber. As described in GB 2175412 and EP 0289545 the exact point in time when a pulse of air is released into the plenum chamber to create a pulse of air through the puff tube, is controlled by a control system (not shown) triggered when an appropriate pattern of light falls on the photodetectors in the sensor chamber 24.

The essential elements of the optical system of FIG. 1 are shown in FIG. 2, where the lenses and field stop making up the eyepiece 16 are denoted as 16A and 16B and 17.

FIG. 3 shows how the arrangement of FIG. 1 can be modified in accordance with the present invention.

In the first place, in FIG. 3 two green LED's 54,56 are located one on each side of the puff tube 30 directed towards the patient's eye 52 and equally spaced from the puff tube and objective lens axis. Although as depicted in FIG. 3 the LED's are shown apparently above and below the puff tube 30, with this orientation of the 45o semi-reflecting surfaces then (for the reasons discussed earlier) they are more preferably mounted to the left and right of the puff tube 30.

The position and spacing of the two LED's 54, 56 are selected so that as the image of the patient's pupil becomes larger than the field of view of the telescope, with continued forward movement of the unit, the operator will see two small green spots which with continued forward movement move apart. Then just as the spots begin to disappear to the left and right of the field of view the red light from 34, 36 which has been reflected from the patient's cornea, begins to appear in the field of view.

The green light spots therefore represent an advance warning that the red segments will shortly appear and if they do not appear symmetrically about the centre of the field of view, the user knows that the unit is not positioned correctly relative to the eye, and can move it accordingly.

An object (shown in FIG. 3B as comprising a pair of cross hairs 60, 62 in a supporting frame or transparent substrate 58) is located downstream of the filter 13 in the lamp housing 12. The position of the object in the support 58 is selected so that the image of the cross hairs 60, 62 comes into focus for the operator at the same distance from the objective lenses to the patient's cornea as gives a correctly aligned and in focus image of the mask 32 onto the plurality of photodetectors 44.

A second object 64 (see FIG. 3A) may be located downstream of 58 containing a single cross hair 66, which will come into focus just before the cross hairs 60, 62.

A third object 70 (see FIG. 3C) containing a different array of cross hairs such as 72, may be located upstream of 58. The visible parts of this object will appear and come into focus if the unit is moved closer to the eye. Continued movement towards the eye can cause the lamp filament to appear and come into focus. Preferably the diameter of the circular wire loop in the array 72 is large enough for parts of it to appear in the two illuminated windows of the mask.

The user can therefore be instructed to look for the cross hair 66 and watch for its replacement by hairs 60, 62 which, when in focus and centred in the field of view, will indicate that the unit should be at the critical distance from the eye 52 for firing to occur. If perchance the hairs 60, 62 are not seen by the user and parts of hair array 72 appear, the user will know to move the unit back, away from the eye, to look for hairs 60, 62.

To make the initial positioning of the tonometer relative to a patient's eye somewhat easier, the eyepiece 16 may be replaced with eyepiece 46 containing a lens 19 having a focal length of the order of 80 mm. Lens 19 forms a simple telescope with the objective lenses 28, 26 which enables the operator to see the patient's eye from a distance. The eyepiece 46 as shown in FIG. 3 also contains a Pechan-Schmidt prism 48 (sometimes called a roof-prism). This presents a correctly orientated and handed image of the patient's face and eye to the user.

When using a modified eyepiece such as 46, a user no longer has to squint along the side of the unit to see if the unit is correctly positioned relative to the eye. Instead the user can now look through the eyepiece and see the face and eyes of a patient at a distance of say 0.5 m. The user can then move the unit so as to centre it on (say) the right eye of the patient and then move forward keeping that eye in the centre of the field of view and centred on the pupil of that eye. As the unit is moved nearer to the eye, the pupil image becomes larger and shortly before or after it fills the field of view so that the latter becomes dark, the reflected green light from the two LED's will break through into the field of view in the form of two green spots, near the centre of the field of view.

Continued forward movement will cause the two green spots to move outwards in opposite directions and disappear, thereafter to be followed by red light which appears as two spaced apart distinct red areas centrally of the field of view and which with continued forward movement enlarge and fill the windows of the mask in the field of view.

As the critical distance from the eye is reached, the black image of the wires 60, 62 of object 58 appear in the otherwise red field of view and come into focus at the precise position at which firing will be triggered. If objects 64 and 70 are also fitted, one of these will appear and come into focus and then go out of focus and disappear just before the wires 60, 62 of 58 appear and come into focus. The wire(s) of the other object will only appear if the unit is moved through the critical position, so as to be too close to the patient's eye. Continued movement of the unit towards the eye will result in the filament of the bulb 34 coming into focus.

If the unit is not centred on the eye, the crossing point of the two wires 60, 62 will not coincide with the centre of the field of view and the wires will appear asymmetrical relative to the field of view. Movement of the unit up or down or sideways to correct this, will find the correct position at which the unit will fire.

Continued movement beyond the point at which the filament comes into focus could result in the puff tube nozzle making contact with the patient's eye, and a further aid to positioning a tonometer (which includes the two LED's but not the eyepiece modification of FIG. 3) relative to the eye under test, is shown at 74 in FIG. 6, with modifications shown in FIGS. 6A and 6B. The device comprises a resiliently deformable extension to the nozzle 20 of the tonometer, whose natural length is just greater than the critical distance between the nozzle and the eye under test, and which can be compressed to a distance equal to and less than the critical distance by pushing against the patient's face, but not so as to allow the leading end of the nozzle to contact the eye.

Thus by positioning the tonometer with the larger end of the extension 74 around the eye to be checked the user can gently push the tonometer towards the patient so as to compress the extension 74, until the light from the two LED's appear (if not already visible) in the field of view. Thereafter the user can move the tonometer forwardly to achieve the firing position, at which point the pulse of air is released towards the eye and the response is monitored by the unit.

In order for the light from the two LED's 54, 56 to reach the cornea whilst aligning the tonometer, the extension 74 may be of transparent material or may be cut away at 76, 78 in the case of the extension 80 in FIG. 3A or may simply comprise a coiled wire helix 82 as shown in FIG. 3B, adapted to fit to the nozzle 20 at the smaller diameter end 84.

Should the user wish to view the eye preparatory to positioning the extension therearound, he/she can do so if the extension is transparent, and if not, by looking through the slots 76, 78 or coil 82.

Preferably the end which is to engage the patient's face around the eye under test is covered with soft crushable material around the loop 86.

The extension may be removable from the nozzle

A covering for the end which is to come into contact with the patient such as 86, may be a disposable item, or may be removable for cleaning or sterilising.

Alternatively, (not shown) the smaller diameter end of the extension 74 (or 80 or 82) is enlarged or shaped so as to encompass the two light sources 54, 56 (or housing enlargements which accommodate them), so that the light from the two sources 54, 56 can pass unimpeded towards the eye 52, whether or not slots 76, 78 are provided, or the extension is in the form of a coil 82.

By carefully selecting the focal length of the eyepiece 16 an image of the wires in the object(s) 58 etc. can be obtained, although the adjustment of the focal length may not be sufficient to form an in focus image of the eye at a distance. However using the extension 74 facilitates the positioning of the unit relative to the eye without the need to see the eye.

It is to be understood that the term lens employed herein can mean a single or multiple element lens.

In addition the colour of the light from the two supplementary light sources may be the same, or different. Thus, if the main source is red, one supplementary source may be green and the other for example yellow or blue.

1.-46. (cancelled).

47. (new) a hand-held air impulse tonometer which includes an objective lens and puff tube assembly and an eyepiece through which a user looks, and comprising a primary light source for directing light through the objective lens assembly towards a patient's eye under test, and further comprising two supplementary light sources located at diametrically opposite points from the optical axis of the objective lens assembly and each arranged to direct light forwardly of the tonometer, such that in use by an operator, and when positioned close to the patient's eye, light from the said two sources, after reflection by the anterior corneal surface of the eye, will be collected by the objective lens assembly and will appear as two small spaced apart areas of light in the user's field of view as seen through the eyepiece, thereby indicating that the tonometer is approaching a critical distance from the eye at which it will discharge a puff of air towards the eye.

48. A tonometer as claimed in claim 47 wherein the spacing and position of the two supplementary light sources relative to the objective lens assembly are selected so that as the tonometer is moved towards an eye under test and begins to approach the distance from the eye at which the tonometer will automatically discharge a puff of air towards the eye, the light reflected by the corneal surface will appear as two closely spaced spots of light which, with continued movement of the unit towards the eye, will begin to move away from each other and then be replaced by two other areas of light as the unit approaches the critical distance from the eye at which the automatic air pulse generating means will be triggered by light from the eye.

49. A tonometer as claimed in claim 48 wherein the position of the two supplementary light sources is selected such that if the tonometer is positioned so that the two small areas of light are symmetrically located about the centre of the field of view, and lie on a straight line passing through that central region of the field of view, the optical axis of the unit is centred on the eye.

50. A tonometer as claimed in claim 47 wherein the wavelength of the light from each supplementary light source is significantly different from that of the primary light source within the tonometer, and the photo-sensors in the tonometer are selected to have a peak response at the wavelength of the light from the said primary source and a minimal or zero response at the wavelength of light from each of the two supplementary light sources, so that any light from the latter which may reach the photo-sensors will not significantly affect the output thereof.

51. A tonometer as claimed in claim 47 which includes a RESET switch which has to be pressed to arm the automatic detection and air puff discharge means, and a further switch which is operated by pressing the RESET switch, which further switch is adapted to supply power to the supplementary light sources, so that on resetting the tonometer, power is provided thereto.

52. A tonometer as claimed in claim 47, wherein the two supplementary light sources comprise two optical fibres leading away from a lamp in the tonometer, to two diametrically opposed positions relative to the tonometer objective lens assembly.

53. A tonometer as claimed in claim 52 wherein the lamp comprises the primary light source in the tonometer, light from which is filtered to provide light to illuminate a mask in the objective lens and puff tube assembly of the tonometer, and to form an image on the photodetectors in the tonometer.

54. A tonometer as claimed in claim 47, wherein the two diametrically opposed points are on opposite sides of the objective lens and puff tube assembly so that the optical path length is the same through the tonometer optics for light from both points, whereby both will be imaged in the same way at the same time.

55. A tonometer as claimed in claim 47, wherein the two light sources are located in lateral enlargements of the tonometer housing on either side of a nozzle containing the objective and puff tube.

56. A tonometer as claimed in claim 47, when modified to further assist the user to determine the position needed to achieve firing of the puff of air towards a patient's eye, wherein an object is placed at a point in the optical path of light from the primary source of light in the tonometer such that an in-focus image of this object will be formed in the user's field of view when the unit is at the critical distance from the patient's eye under test at which the automatic air discharge means will be triggered.

57. A tonometer as claimed in claim 56 wherein a second object is located close to the first object, in a plane which is spaced from the plane containing the first object, such that an image of the second object will come into focus in the user's of view just before the image of the first object comes into focus, as the unit is moved towards the patient's eye.

58. A tonometer as claimed in claim 57 wherein a third object is provided, also visually distinguishable from both the first and second objects and at a position relative to the primary light source such that its image will come into focus if the unit is moved closer to the eye than the critical distance from the eye under test at which the automatic air discharge means will be triggered.

59. A tonometer as claimed in claim 47, wherein the eyepiece magnification is selected so as to produce an in-focus image of the patient's eye to the user in the user's field of view when at or beyond a given distance from the tonometer, but which at shorter distances from the eye, but greater than that at which an automatic air discharge means in the tonometer will be triggered, will no longer produce an in-focus image of the eye, whereby the field of view becomes less than the area of the pupil of the patient's eye, so that the field of view becomes dark, thereby enabling the light from the two supplementary light sources clearly to be seen as the tonometer moves closer to the critical position at which the air-puff is to be discharged.

60. A tonometer as claimed in claim 47, wherein the objective lens and puff tube assembly is contained with a nozzle and a resiliently deformable extension is provided to the nozzle, whose natural length is greater than the critical distance between the nozzle and the eye under test at which firing will occur, and which can be compressed to bring the tonometer to the critical distance at which the automatic air discharge means is triggered, by pushing against the patient's face.

61. A method of aligning and positioning relative to a patient's eye a tonometer as claimed in claim 47, wherein a user looks through the eyepiece towards the patient's eye which is to be tested, and while continuing to look through the eyepiece moves the tonometer towards the patient, along a path which keeps the optical axis of the tonometer objective centred on the eye to be tested, and adjusts the tonometer relative to the patient's eye so that light from the two supplementary light sources reflected off the patient's eye appears as two spots of light symmetrically and midway of the field of view, and which diverge as the tonometer is moved closer to the eye, until light from the primary light source in the tonometer projected from the tonometer towards the patient's eye and reflected therefrom, replaces the first two spots of light in the user's field of view and illuminates the windows of the mask as the tonometer approaches the position relative to the patient's eye at which the tonometer will automatically discharge a puff of air towards the eye, thereby to provide an indication to the user that the tonometer is very close to the position at which the puff of air will be discharged.

62. A method of measuring the intra-ocular pressure of a patient's eye using a tonometer as claimed in claim 47, wherein the user looks through the eyepiece towards the patient's eye to be tested, and while continuing to look through the eyepiece moves the tonometer towards the patient along a path which keeps the optical axis of the tonometer objective centred on the eye to be tested, and adjusts the tonometer relative to the patient's eye so that light from two supplementary light sources reflected off the patient's eye appears as two spots of light symmetrically and midway of the field of view and which diverge as the tonometer is moved closer to the eye, until light from the primary source of light in the tonometer, projected from the tonometer towards the eye under test, and reflected therefrom back towards the tonometer replaces the first two spots of light in the user's field of view and illuminates windows in a mask in the objective lens assembly as the tonometer gets close to the position at which the pneumatic air puff generating system of the tonometer will be automatically triggered to discharge a puff of air towards the eye and continuing to move the tonometer towards the eye to trigger the discharge, after which a numerical value proportional to the intra-ocular pressure of the eye is computed from a variation in the light reflected by the eye and received by the photodetectors of a detector in the tonometer as the eye is momentarily distorted by the force of the puff of air.

63. A method of measuring the intra-ocular pressure of a patient's eye using a tonometer as claimed in claim 60, wherein a user positions the tonometer with the extension in contact with the patient's face around the eye to be tested, and thereafter looks through the eyepiece towards the patient's eye, and while continuing to look through the eyepiece continues to move the tonometer towards the patient's face, thereby compressing the extension until the pneumatic air puff generating system of the tonometer is automatically triggered to discharge a puff of air towards the eye, after which a numerical value proportional to the intra-ocular pressure of the eye is computed by a computing device in the tonometer from signals indicative of a variation in the light reflected by the eye and received by photodetectors of a detector in the tonometer as the eye is momentarily distorted by the force of the puff of air.
